(19) United States
(12) Patent Application Publication
(10) Pub. No.: US 2015/0246067 A1
(43) Pub. Date: Sep. 3, 2015

(54) ANTIVIRAL TREATMENT OF LYMPHOMA AND CANCER

(71) Applicant: THE REGENTS OF THE UNIVERSITY OF MICHIGAN, Ann Arbor, MI (US)

(72) Inventors: Mark H. Kaplan, Ann Arbor, MI (US); Rafael Contreras-Galindo, Ann Arbor, MI (US); David Markovitz, Ann Arbor, MI (US)

(21) Appl. No.: 14/640,992

(22) Filed: Mar. 6, 2015

Related U.S. Application Data
(63) Continuation of application No. 13/321,416, filed on Feb. 13, 2012, now abandoned.
(60) Provisional application No. 61/180,321, filed on May 21, 2009.

(51) Int. Cl. A61K 31/708; A61K 31/675; A61K 31/52; A61K 31/536; A61K 31/551; C12Q 1/70; A61K 31/427; A61K 31/496; A61K 31/635; A61K 31/7068; A61K 31/513; A61K 31/7072; A61K 31/4418

(52) U.S. Cl. CPC A61K 31/708; A61K 31/7072; A61K 31/675; A61K 31/52; A61K 31/536; A61K 31/551; A61K 31/4418; A61K 31/427; A61K 31/496; A61K 31/635; A61K 31/7068; A61K 31/513; C12Q 1/703; C12Q 2600/136; C12Q 2600/158

(57) ABSTRACT

The present invention provides compositions and methods to treat lymphoma and cancer. In particular, the present invention provides treatment of lymphoma and cancer using anti-HERV-K(HML-2) therapies. The present invention further provides compositions and methods for characterizing patient samples to, for example, select or identify therapeutic options or assess the impact of therapies.

### CROSS REFERENCE TO RELATED APPLICATIONS

This application claims priority to U.S. Provisional Patent Application Ser. No. 61/180,321 filed May 21, 2009, hereby incorporated by reference in its entirety.

### FIELD OF THE INVENTION

The present invention provides compositions and methods to treat lymphoma and cancer. In particular, the present invention provides treatment of lymphoma and cancer using anti-HERV-K(HML-2) therapies. The present invention further provides compositions and methods for characterizing patient samples to, for example, select or identify therapeutic options or assess the impact of therapies.

### BACKGROUND OF THE INVENTION

Non Hodgkin's lymphoma (NHL) has an annual incidence of approximately 12.8 cases/year/100000 persons from 2000-2003 as compared to breast cancer at 82.7, prostate cancer at 60, lung cancer at 27.2 and colorectal cancer at 20.5 cases/year/100,000 people. In individuals with HIV infection from 1992-1995 the incidence of NHL was 1011.8 cases/100000 HIV patients/year or 59.5 times higher than the general population but this incidence has fallen dramatically to 212.5 cases/year/100,000 HIV infected patients from 2000-2003 (16.6 times higher than the general population). Central nervous system lymphoma and diffuse large B cell lymphoma have been most dramatically affected. This fall in incidence is due mostly to the advent of highly active antiretroviral therapy (HAART) and represents one of the greatest triumphs in cancer prevention in modern medicine. In addition to reduction in incidence of lymphoma, HAART has allowed better survival of patients when they are treated with chemotherapy so that in some studies the survival of HIV infected patients from DHBCL is almost as good as in non HIV patients.

Epstein Barr Virus (EBV) and Human Herpes Virus-8 (HHV-8) have been postulated to be the principal viral agents associated with HIV associated lymphomas. EBV is found in almost 100% of central nervous system lymphoma and is present in most cases of diffuse large B cell lymphoma (DLBCL) with immunoblastic morphology. EBV is present in over 60% of Burkitt's lymphoma and most cases of HIV associated Hodgkin's lymphoma. The recently discovered virus HHV 8 has been found in all cases of Kaposi's sarcoma. In the rare cases of primary effusion lymphomas (PEL) and its solid variant plasmablastic lymphoma (PBL) of the oral cavity 100% of tumor cells carry multiple copies of HHV8 in addition to carrying EBV in up to 90% of tumors. HHV8 is also present in 100% of large B cell lymphoma arising in Kaposi's sarcoma-associated herpes virus (KSHV) associated multicentric Castleman's disease. In this rare lymphoma the KSHV infected B cells have a pre plasma cell phenotype and plasmacytic/plasmablastic morphology. In spite of the association of these viruses with the above HIV associated lymphomas, these two gamma herpes viruses cannot account for over 60% of DLBCL which lack immunoblastic plasmacytoid features (which are the most common lymphomas occurring in HIV) and over 30% of HIV associated Burkitt's lymphoma. In non HIV infected patients including the most common lymphomas notably DLBCL and follicular lymphoma, EBV and HHV 6 are uncommonly found except possibly in some Burkitt's lymphoma where it is found only in patients from epidemic areas and is often absent in sporadic BL.

With the sequencing of the human genome it is apparent that over 8% of the human genome is composed of retroviral elements. HERV K (HML2) appears to be one of the most recent elements to have entered the primate genome having its first entry estimated to be about 30,000,000 years ago. This virus has made multiple subsequent entries with the last being proposed to be about 200,000 years ago. Fully intact HERV K (HML2) DNA is present in about 52 different chromosomal locations. Most of these elements have developed deleterious mutations in gag, pol and env rendering them unable to replicate. However, some have intact gag, some an intact pol and some an intact env.

HERV K (HML2) exists in 2 forms, type 1 and type 2. The type 1 viruses have a 292 base pair deletion in env which prevents these viruses from making competent envelopes but these virions can produce a regulatory protein called Np9 which has oncogenic properties. Some have intact pol and gag sequences. The type 2 virus have no such deletion and they are able to make envelope protein. The type 2 virus is found in approximately 10 different chromosomal locations in the human genome. Two HERV K (HML2) family members notably HERV K 113 and K115 possess a complete set of viral genes with intact open reading frames which are insertionally polymorphic in man and are probably the most recent HERV K (HML2) entries into the human genome. Type 2 virus also produces a regulatory protein called Rec. Rec is a 14,000 base pair protein which is similar to HIV Rev. This protein acts as a chaperone for mRNA generated in the nucleus to conduct it through the nuclear pore where it can be transcribed into protein. Replicating virions of HERV K (HML2) should produce Rev and this can induce antibody in patients if virus is activated.

There is growing interest in these viruses to search for active forms which might still have capacity to replicate either by a fully competent virus that may have entered the human genome even more recently than K113 and K115, and/or from some virus which emerges as a fully competent infectious virus through recombination and or through complementation from the wide variety of HERV K (HML2) insertions in the genome.

Viral particles can be produced by HERV K (HML2) and these were first seen in teratocarcinoma cell lines and antibody to HERV K (HML2) has been demonstrated in some patients with teratocarcinoma. Many breast cancer cell lines produce these particles but how they are linked to breast cancer is not yet known. Recently HERV K (HML2) viral antigens have been demonstrated in malignant melanoma skin biopsies and lymph node metastases and viral particles can be seen in melanoma cell lines. These patients also have antibody present to HERV K (HML2) viral antigens and the higher titers appear to be associated with more wide spread metastatic disease. These viruses appear linked in some way to neoplastic disease.

To better understand how these viruses might replicate, two laboratories have reconstructed full length HERV K (HML2) viral clones with CMV promoters called the “Phoenix virus” and HERV Kcon and have shown that these reconstituted viruses have capacity to replicate. In patients with HIV, HERV K (HML2) viral RNA can be found in the plasma of HIV patients at high concentration. Furthermore, in both HIV and non HIV associated lymphoma patients, there is a dramatic increase in the HERV K (HML2) viral RNA present in the plasma of these patients. Free HERV K (HML2) viral particles can be visualized in plasma by immune electron microscopy. These particles have the appropriate density for a retrovirus and have packaged both gag and env proteins as demonstrated by western blot.

### SUMMARY

In some embodiments, the present invention comprises a method for treating cancer comprising treating a subject suffering from cancer with one or more compounds sufficient to reduce the viral load of HERV K (HML-2). In some embodiments of the present invention, a subject suffers from lymphoma. In some embodiments of the present invention, a subject suffers from HIV-associated lymphoma. In some embodiments of the present invention, a subject suffers from non-HIV-associated lymphoma. In some embodiments of the present invention, compounds comprise antiretroviral pharmaceuticals. In some embodiments of the present invention, antiretroviral pharmaceuticals comprise reverse transcriptase inhibitors. In some embodiments, reverse transcriptase inhibitors are selected from nucleoside analog reverse transcriptase inhibitors, nucleotide analog reverse transcriptase inhibitors, and non-nucleoside reverse transcriptase inhibitors. In some embodiments, reducing the viral load of HERV K (HML-2) causes a reduction in tumor burden.

In some embodiments, the present invention provides a method of screening compounds useful in the treatment of cancer comprising screening compounds for activity in reducing viral load of HERV K (HML-2). In some embodiments, the screening is performed in vitro. In some embodiments, the screening is performed in vivo. In some embodiments, the screening comprises administering one or more compounds to cells and assaying cells for a reduction in viral load of HERV K (HML-2). In some embodiments, the screen comprises high throughput screening. In some embodiments, compounds are further assayed for usefulness in treating cancer. In some embodiments, cancer comprises lymphoma. In some embodiments, lymphoma comprises HIV-associated lymphoma. In some embodiments, lymphoma comprises non-HIV-associated lymphoma. In some embodiments, compounds comprise antiretroviral pharmaceuticals. In some embodiments, antiretroviral pharmaceuticals comprise reverse transcriptase inhibitors. In some embodiments, reverse transcriptase inhibitors are selected from nucleoside analog reverse transcriptase inhibitors, nucleotide analog reverse transcriptase inhibitors, and non-nucleoside reverse transcriptase inhibitors.

In some embodiments, the presence of, amount of, or type of (e.g., sequence of) HERV K in a subject is identified to characterize a subject. This information may be used to select or monitor a therapy or other intervention. In some embodiments, HERV K is analyzed prior to therapy (i.e., test then treat). In some embodiments, HERV K may further be analyzed during or following treatment (e.g., test/treat/test or treat/test). In some embodiments, therapy is altered following testing (e.g., test/treat/test/treat or treat/test/treat). Various combinations of treatment and assessment of HERV K status are contemplated by the invention.

### BRIEF DESCRIPTION OF THE DRAWINGS

The foregoing summary and detailed description is better understood when read in conjunction with the accompanying drawings which are included by way of example and not by way of limitation.

FIG. 1 shows a graph depicting the correlation between HERV K (HML-2) type 2 RNA load and large B-cell lymphoma.

FIG. 2 shows a graph depicting the correlation between HERV K (HML-2) type 2 RNA load and follicular lymphoma.

FIG. 3 shows a graph depicting a reduction in HERV K (HML-2) type 2 viral load upon cancer remission.

FIG. 4 shows reduction of reverse transcriptase activity upon treatment of cells with antiretrovirals.

FIG. 5 shows reduction of reverse transcriptase activity and HERV K (HML-2) type 2 viral load upon treatment of cells with antiretrovirals.

FIG. 6 shows a graph depicting the effect of AZT and PFA on NCCIT cells.

### DEFINITIONS

To facilitate an understanding of the present invention, a number of terms and phrases are defined below:

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

In some embodiments, a subject is selected from a group consisting of subject at risk for developing cancer, a subject suspected of having cancer, a subject suspected of having cancer metastasis, a subject suspected of having cancer recurrence, a subject known to have cancer, a subject undergoing cancer therapy, and a subject that has completed cancer therapy.

As used herein, the term “subject suspected of having cancer” refers to a subject that presents one or more symptoms indicative of a cancer) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass or increased PSA level, breast cancer or lymphoma biopsy, leukemic cells in the circulation or marrows), but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing cancer tissue in a subject” refers to the identification of one or more properties of a cancer tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer markers, including but not limited to, the cancer markers disclosed herein.

As used herein, the term “cancer marker” refers to any biologic compound, molecule, macromolecule, or complex (e.g. virus (e.g. HERV-K (HML-2)) whose presence or level, alone or in combination with other factors is correlated with cancer or prognosis of cancer. The correlation may relate to either an increased or decreased expression or production. For example, the presence of viral particles ((e.g. HERV-K (HML-2)) may be indicative of cancer, or lack of expression of a gene may be correlated with poor prognosis in a cancer patient. The “cancer marker” may be correlated with cancer or may be causative.

As used herein, the term “cancer marker genes” refers to a gene or genes whose presence or expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient.

As used herein, the term “a reagent that specifically detects the presence or absence of HERV-K(HML-2) target” refers to reagents used to detect the presence of or expression of one or more HERV-K(HML-2) targets (e.g., including but not limited to, the cancer markers of the present invention). Examples of suitable reagents include but are not limited to, nucleic acid probes capable of specifically hybridizing to the HERV-K(HML-2) targets of interest, PCR primers capable of specifically amplifying the gene of interest, and antibodies capable of specifically binding to proteins expressed by the gene of interest. Other non-limiting examples can be found in the description and examples below.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “initial diagnosis” refers to results of initial cancer diagnosis (e.g. the presence or absence of cancerous cells). An initial diagnosis does not include information about the stage of the cancer of the risk.

As used herein, the term “biopsy tissue” refers to a sample of tissue that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands.

The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization[1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42.degree. C. in a solution consisting of 5.times.SSPE (43.8 g/l NaCl, 6.9 g/l NaH₂PO₄H₂O and 1.85 g/1 EDTA, pH adjusted to 7.4 with NaOH), 0.5% SDS, 5.times.Denhardt's reagent and 100 .mu.g/ml denatured salmon sperm DNA followed by washing in a solution comprising 0.1.times.SSPE, 1.0% SDS at 42.degree. C. when a probe of about 500 nucleotides in length is employed.

“Medium stringency conditions” when used in reference to nucleic acid hybridization comprise conditions equivalent to binding or hybridization at 42.degree. C. in a solution consisting of 5.times.SSPE (43.8 g/1 NaCl, 6.9 g/l NaH₂PO₄H₂O and 1.85 g/1 EDTA, pH adjusted to 7.4 with NaOH), 0.5% SDS, 5.times.Denhardt's reagent and 100. mu.g/ml denatured salmon sperm DNA followed by washing in a solution comprising 1.0.times.SSPE, 1.0% SDS at 42.degree. C. when a probe of about 500 nucleotides in length is employed.

“Low stringency conditions” comprise conditions equivalent to binding or hybridization at 42.degree. C. in a solution consisting of 5.times.SSPE (43.8 g/1 NaCl, 6.9 g/l NaH₂PO₄H₂O and 1.85 g/1 EDTA, pH adjusted to 7.4 with NaOH), 0.1% SDS, 5.times.Denhardt's reagent [50.times.Denhardt's contains per 500 ml: 5 g Ficoll (Type 400, Pharamcia), 5 g BSA (Fraction V; Sigma)] and 100. mu.g/ml denatured salmon sperm DNA followed by washing in a solution comprising 5.times.SSPE, 0.1% SDS at 42.degree. C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and non-blood products, for example, urine, spinal fluid, bile, saliva, stool, tears, sweat, mucous, semen, cells, and tissues, and the like. Environmental samples include environmental material such as surface matter, soil, water, and industrial samples. Such examples are not however to be construed as limiting the sample types applicable to the present invention.

### DETAILED DESCRIPTION OF EMBODIMENTS

The present invention provides methods to treat and diagnose lymphoma and cancer. In particular, the present invention provides treatment of lymphoma and cancer using anti-HERV-K (HML-2) therapies. Accordingly, the present invention provides methods, reagents, and kits for the detection of markers, drug screening, and therapeutic applications. In some embodiments, HERV-K(HML-2) is a cancer marker (e.g. marker of lymphoma). In some embodiments, HERV-K(HML-2) is a cancer causative agent.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The human genome harbors numerous retroviral sequences that comprise up to 8% of the host genome, many of which have accumulated lethal mutations that have impaired their ability to replicate. (Nelson et al. Mol Pathol 2003; 56:11-18; Wang-Johanning et al. Oncogene 2003; 22:1528-1535; Hughes et al. Proc Natl Acad Sci USA 2004; 101: 1688-1672). The human endogenous retrovirus type-K (HERV-K.HML-2) family is represented by many proviruses, some of which possess intact open reading frames (ORFs) for gag, prt, pol, and env genes. (Barbulescu et al. Curr Biol 1999; 9:861-868; Paces et al. Nucleic Acids Res 2002; 30:205-206). HERV-K(HML-2) is an endogenous retroviral subfamily with the ability to produce viral particles. (Bannert et al. Proc Natl Acad Sci USA 2002; 101 Suppl 2:14572-14579; Simpson et al. Virology 1996; 222:451-456; Bieda et al. J Gen Virol 2001; 3:591-596; Boller et al. Virology 1993; 1:349-353). However, an intact HERV-K proviral sequence (K113) and perhaps other unidentified unfixed elements may code for replication-competent viruses. (Turner et al. Curr Biol 2001; 11:1531-1535; Moyes et al. Genomics 2005; 86:337-341; Bleshaw et al. J Virol 2005; 79: 12507-12514). The detection of anti-HERV-K antibodies in the plasma of 70% of HIV-1 patients compared to only 3% of healthy blood donors. (Lower et al. Proc Natl Acad Sci USA 1996; 93:5177-5184). Antibodies to HERV-K were also detectable in drug users, but only after HIV-1 seroconversion. (Vogetseder et al. AIDS Res Hum Retroviruses 1993; 9:687-694). U.S. Patent Application 20080261216, herein incorporated by reference in its entirety, describes that it was found that if HERV-K viral particles are made, they may be protected by viral envelopes in plasma of HIV-1 infected individuals, and that the RNA genome is directly amplified from viral RNA extractions of plasma. All of the above references are herein incorporated by reference in their entireties.

In some embodiments, the present invention provides therapies for cancer and cancer-related illnesses (e.g. Acute Lymphoblastic Leukemia, Acute Myeloid Leukemia, Adrenocortical Carcinoma, AIDS-Related Cancers, AIDS-Related Lymphoma, Anal Cancer, Appendix Cancer, Astrocytoma, Atypical Teratoid/Rhabdoid Tumor, Basal Cell Carcinoma, Bile Duct Cancer, Bladder Cancer, bone cancer (e.g. Osteosarcoma or Malignant Fibrous Histiocytoma), Brain Stem Glioma, Brain Tumor (e.g. Adult, Childhood, Brain Stem Glioma, Atypical Teratoid/Rhabdoid Tumor, Embryonal Tumors, Cerebellar Astrocytoma, Cerebral Astrocytoma, Malignant Glioma, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma, Visual Pathway and Hypothalamic Glioma, Brain and Spinal Cord Tumors), Breast Cancer, Bronchial Tumors, Burkitt Lymphoma, Carcinoid Tumor, Carcinoma, Atypical Teratoid/Rhabdoid Tumor, Embryonal Tumors, Central Nervous System Lymphoma, Cerebellar Astrocytoma, Cervical Cancer, Childhood Cancers, Chordoma, Chronic Lymphocytic Leukemia, Chronic Myelogenous Leukemia, Chronic Myeloproliferative Disorders, Colon Cancer, Colorectal Cancer, Craniopharyngioma, Cutaneous T-Cell Lymphoma, Embryonal Tumors, Endometrial Cancer, Ependymoblastoma, Ependymoma, Esophageal Cancer, Ewing Family of Tumors, Extracranial Germ Cell Tumor, Extragonadal Germ Cell Tumor, Extrahepatic Bile Duct Cancer, Eye Cancer (e.g. Intraocular Melanoma, Retinoblastoma, etc.), Gallbladder Cancer, Gastric (Stomach) Cancer, Gastrointestinal Carcinoid Tumor, Gastrointestinal Stromal Tumor (GIST), Germ Cell Tumor (e.g. Extracranial, Extragonadal, Ovarian, etc.), Gestational Trophoblastic Tumor, Glioma (e.g., Adult, Childhood, Brain Stem, Cerebral Astrocytoma, Visual Pathway and Hypothalamic, etc.), Hairy Cell Leukemia, Head and Neck Cancer, Hepatocellular (Liver) Cancer, Hodgkin Lymphoma, Hypopharyngeal Cancer, Hypothalamic and Visual Pathway Glioma, Intraocular Melanoma, Islet Cell Tumors (Endocrine Pancreas), Kaposi Sarcoma, Kidney (Renal Cell) Cancer, Laryngeal Cancer, Leukemia (e.g. Acute, Lymphoblastic, Adult, Childhood, Acute Myeloid, Chronic Lymphocytic, Chronic Myelogenous, Hairy Cell, etc.), Lip and Oral Cavity Cancer, Liver Cancer, Lung Cancer (e.g. Non-Small Cell, Small Cell, etc.), Lymphoma (e.g. AIDS-Related, Burkitt, Cutaneous T-Cell, Mycosis Fungoides, Sézary Syndrome, Hodgkin, Adult, Childhood, Non-Hodgkin, Primary Central Nervous System, etc.), Macroglobulinemia, Malignant Fibrous Histiocytoma of Bone and Osteosarcoma, Medulloblastoma, Medulloepithelioma, Melanoma, Merkel Cell Carcinoma, Mesothelioma, Metastatic Squamous Neck Cancer, Mouth Cancer, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia (e.g. Chronic, Acute, etc.), Myeloid Leukemia, Myeloma, Myeloproliferative Disorders, Nasal Cavity and Paranasal Sinus Cancer, Nasopharyngeal Cancer, Neuroblastoma, Oral Cancer, Oropharyngeal Cancer, Osteosarcoma and Malignant Fibrous Histiocytoma of Bone, Ovarian Cancer (e.g. Childhood, Ovarian Epithelial Cancer, Ovarian Germ Cell Tumor, Ovarian Low Malignant Potential Tumor, etc.), Pancreatic Cancer, Islet Cell Tumors, Papillomatosis, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System Lymphoma, Prostate Cancer, Rectal Cancer, Renal Cell (Kidney) Cancer, Renal Pelvis and Ureter, Respiratory Tract Carcinoma Involving the NUT Gene on Chromosome 15, Retinoblastoma, Rhabdomyosarcoma, Salivary Gland Cancer Sarcoma, (e.g. Ewing Family of Tumors, Kaposi, Soft Tissue, Adult, childhood, Uterine, etc.), Sézary Syndrome, Skin Cancer (e.g. Nonmelanoma, Childhood, Melanoma, Carcinoma, Merkel Cell, etc.) Small Intestine Cancer, Soft Tissue Sarcoma, Squamous Cell Carcinoma, Squamous Neck Cancer with Occult Primary, Stomach (Gastric) Cancer, Supratentorial Primitive Neuroectodermal Tumors, T-Cell Lymphoma, Testicular Cancer, Throat Cancer, Thymoma and Thymic Carcinoma, Thyroid Cancer, Transitional Cell Cancer of the Renal Pelvis and Ureter, Trophoblastic Tumor, Unknown Primary Site, Unusual Cancers of Childhood Ureter and Renal Pelvis, Urethral Cancer, Uterine Cancer (e.g. Endometrial, Uterine Sarcoma, etc.), Vaginal Cancer, Visual Pathway and Hypothalamic Glioma, Vulvar Cancer, Waldenström Macroglobulinemia, Wilms Tumor, etc.).

In some embodiments of the present invention, pharmaceutical compositions are used for the treatment of cancer. In some embodiments of the present invention, pharmaceutical compositions are used for the treatment of viral infection (e.g. HERV-K(HML-2)). In some embodiments of the present invention, pharmaceutical compositions are used for the treatment of cancer by the reduction of retroviral load (e.g. HERV-K(HML-2)). Within such methods, the pharmaceutical compositions described herein are administered to a patient, typically a warm-blooded animal (e.g. a human). A patient may or may not be afflicted with cancer. Accordingly, the above pharmaceutical compositions may be used to prevent the development of a cancer or to treat a patient afflicted with a cancer. A patient may or may not have circulating viral particles (e.g. HERV-K(HML-2) particles). Accordingly, the above pharmaceutical compositions may be used to prevent the spread or production of a viral particles (e.g. HERV-K(HML-2)) or to treat a patient afflicted with viral particles (e.g. HERV-K(HML-2)). In some embodiments, a patient treated by the present invention is not infected with HIV (e.g. HIV-1, HIV-2, etc.). Pharmaceutical compositions and vaccines may be administered either prior to or following surgical removal of primary tumors and/or treatment such as administration of radiotherapy or conventional chemotherapeutic drugs. As discussed herein, administration of the pharmaceutical compositions may be by any suitable method, including administration by intravenous, intraperitoneal, intramuscular, subcutaneous, intranasal, intradermal, anal, vaginal, topical and oral routes.

In some embodiments, the present invention provides therapies that kill cancer cells, induce apoptosis in cancer cells, stop or slow the spread of cancer, stop or reduce cancer metastasis, stop or reduce tumor formation, reduce tumor load, minimize the effects of cancer, support the ability of the body to fight cancer, and/or serve as an antagonist to cancer, cancer cells, or cancer-related diseases. In some embodiments, the compounds act as a cancer therapy by directly or indirectly targeting a cancer marker (e.g. HERV-K(HML-2)). In some embodiments, the present invention provides methods, regents, and kits that are cancer therapies. In some embodiments, the present invention treats cancer (e.g. lymphoma) by reducing or eliminating the viral load (e.g. one of more retroviruses (e.g. HERV-K(HML-2))) within a subject.

In some embodiments, one or more retroviruses and/or retroviral elements (e.g. HERV-K(HML-2)) are a cause of, the cause of, a contributing factor to, and/or an aggravating factor to cancer (e.g. breast cancer, lymphoma, etc.) and/or cancer-related illnesses. In some embodiments, reducing the viral load of HERV-K(HML-2) and/or other retroviruses provides a cancer therapy (e.g. killing cancer cells, reducing tumor load, etc.). In some embodiments, HERV-K(HML-2) viral proteins are expressed from exogenous genes (e.g. genes which have infected a subject). In some embodiments, HERV-K(HML-2) viral proteins are expressed from endogenous genes (e.g. viral protein genes which are integrated into the subject's genome). In some embodiments, HERV-K(HML-2) viral proteins expressed within a subject are capable of assembling into a viral element (e.g. virion, virus, mature virus, viral particle, etc.). In some embodiments, viral elements (e.g. virion, virus, mature virus, viral particle, etc.) produced and assembled within a subject are capable of reinfecting the subject, infecting another subject, reproducing, and/or replicating. In some embodiments, HERV-K(HML-2) viral proteins expressed within a subject are not capable of assembling into a viral element (e.g. virion, virus, mature virus, viral particle, etc.). In some embodiments, viral elements (e.g. virion, virus, mature virus, viral particle, etc.)

produced and assembled within a subject are not capable of reinfecting the subject, infecting another subject, reproducing, and/or replicating. In some embodiments, viral proteins (e.g. HERV-K(HML-2) viral proteins) are expressed from one or more endogenous genes within a subject's genome.

In some embodiments, the present invention provides one or more antiviral therapies. In some embodiments, compounds of the present invention inhibit one or more retroviruses or retroviral elements (e.g. HIV-1, HIV-2, HERV-K(HML-2, etc.). As one skilled in the art will appreciate, the compounds of the present invention may inhibit a variety of retroviruses, retroviral elements, and may inhibit viruses, other than retroviruses. Compounds which inhibit one or more of the following may also find utility in the present invention: HERV-K(HML-2) type 1, HERV-K(HML-2) type 2, Type C and Type D retroviruses, HTLV-1, HTLV-2, HIV, FLV, SIV, MLV, BLV, BIV, equine infections, anemia virus, avian sarcoma viruses, such as Rous sarcoma virus (RSV), hepatitis type A, B, non-A and non-B viruses, arboviruses, varicella viruses, measles, mumps, rubella viruses, etc. In some embodiments, the present invention provides antiviral therapies in doses and/or combinations which are not useful (or are sub-optimally useful) as therapies against HIV (e.g. removal of a pharmaceutical form a combinatorial therapy (e.g. removal of a fusion inhibitor from a multi-drug antiviral therapy, or removal of a protease from a multi-drug antiviral therapy), replacement of a pharmaceutical in a combinatorial therapy, or a dose which would be ineffective or not commonly used in treating HIV). In some embodiments, the present invention provides antiviral therapies in doses and/or combinations which are not preferred as a therapy against HIV. In some embodiments, the treatment regimens of the present invention differ from the most effective HIV treatment regimens (Robbins et al. 2003, N Engl J Med, 349; 24., Shafer et al. 2003, N Engl J Med, 349; 24, herein incorporated by reference in their entireties) in one or more ways (e.g. dose, combination of drugs, etc.). In some embodiments, treatments of the present invention find utility in treating HIV-infected subject and/or non-HIV-infected subjects.

In some embodiments, the present invention provides antiretroviral drugs comprising one or more of, but not limited to, reverse transcriptase inhibitors, nucleoside analog reverse transcriptase inhibitors (e.g. Zidovudine, Didanosine, Zalcitabine, Stavudine, Lamivudine, Abacavir, Emtricitabine, Atricitabine, etc.), nucleotide analog reverse transcriptase inhibitors (e.g. Tenofovir, Adefovir, etc.), non-nucleoside reverse transcriptase inhibitors (e.g. Efavirenz, Nevirapine, Delavirdine, Etravirine, etc.), protease inhibitors (e.g. Saquinavir, Ritonavir, Indinavir, Nelfinavir, Amprenavir, Lopinavir, Atazanavir, Fosamprenavir, tipranavir, Darunavir, etc.), fusion inhibitors (e.g. Maraviroc, Enfuvirtide, etc.), integrase inhibitors (e.g. Raltegravir, Elitegravir, etc.), entry inhibitors (e.g. Maraviroc, Enfuvirtide, etc.), maturation inhibitors (e.g. Bevirimat, etc.), portmanteau inhibitors, etc. In some embodiments, the present invention provides any compounds that function as an antiretroviral (e.g. AZT (Zidovudine), FTC (Emtricitabine), 3TC (Lamivudine), ddC (zalcitabine), d4T (Stavudine), ddI (Dideoxyinosine), TDF (Tenofovir disoproxyl fumarato), ABC (Abacavir), β-d hydroxy cytidine, Efavirenz, Nevirapine, Etravirine, Atazanavir, Ritonavir, Indinavir, Amprenavir, etc.). In some embodiments, the present invention provides antiretroviral therapies that include administration of one or more pharmaceutical compounds (e.g. 1 compound, 2 compounds, 3 compounds, 4 compounds, 5 compounds, 6 compounds, 7 compounds, 8 compounds, 9 compounds, 10 compounds, >10 compounds). In some embodiments, the present invention provides combination therapy in which two or more compounds are simultaneously administered or administered in sequence. In some embodiments, the present invention provides highly active antiretroviral therapy (HAART) or the administration of a plurality of different antiretroviral drugs in combination to overwhelm the ability of a retrovirus to develop resistance to a single therapy. In some embodiments, the present invention provides a regimen involving administration of one or more approaches including but not limited to antiretrovirals, cancer chemotherapy, radiation, diet, exercise, surgery, nutrition, supplementation, etc. In some embodiments, one or more antiretroviral therapies (e.g. one or more pharmaceuticals) are administered in combination with one or more cancer therapies (e.g. chemotherapy, radiation, etc.).

In some embodiments, the present invention provides drug screening assays (e.g., to screen for anticancer drugs). The screening methods of the present invention utilize cancer markers identified using the methods of the present invention (e.g., including but not limited to, HERV-K(HML-2) targets). For example, in some embodiments, the present invention provides methods of screening for compounds that alter (e.g., decrease) the production of cancer markers. The compounds or agents may interfere with transcription. The compounds or agents may interfere with mRNA produced from: HERV-K(HML-2) (e.g., by RNA interference, antisense technologies, etc.). The compounds or agents may interfere with pathways that are upstream or downstream of the biological activity of the HERV-K(HML-2) target. In some embodiments, candidate compounds are antisense or interfering RNA agents (e.g., oligonucleotides) directed against cancer markers (e.g. HERV-K(HML-2). In some embodiments, compounds or agents may interfere with HERV-K(HML-2) replication. In other embodiments, candidate compounds are antibodies or small molecules that specifically bind to a cancer marker regulators or expression products of the present invention and inhibit its biological function.

In one screening method, candidate compounds are evaluated for their ability to alter cancer marker production by contacting a compound with a cell producing a cancer marker and then assaying for the effect of the candidate compounds on expression. In some embodiments, the effect of candidate compounds on production of a cancer marker is assayed for by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention, have an inhibitory (or stimulatory) effect on, for example, cancer marker production or cancer marker activity, or have a stimulatory or inhibitory effect on, for example, the expression or activity of a cancer marker substrate. Compounds thus identified can be used to modulate the activity of target gene products (e.g., cancer marker genes (e.g. (e.g., HERV-K(HML-2) gene or genes)) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly lymphoma, leukemia and breast cancer.

In one embodiment, the invention provides assays for screening candidate or test compounds that are substrates of a cancer marker protein or polypeptide or a biologically active portion thereof (e.g., HERV-K(HML-2) proteins). In another embodiment, the invention provides assays for screening candidate or test compounds that bind to or modulate the activity of a cancer marker protein or polypeptide or a biologically active portion thereof (e.g., HERV-K(HML-2) proteins). In some embodiments, the invention provides assays for screening candidate or test compounds that are inhibitors of viral replication (e.g. retroviral replication (e.g. HERV-K(HML-2) replication)). In another embodiment, the invention provides assays for screening candidate or test compounds that bind to or modulate the effects of viruses (e.g. retroviruses (e.g. HERV-K(HML-2))), spread of viruses, expression of viral proteins, etc.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the one-bead one-compound library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In some embodiments, an assay is a cell-based assay in which a cell that expresses a cancer marker mRNA or protein, a biologically active portion thereof, or a viral particle cancer marker (e.g. HERV-K(HML-2)) is contacted with a test compound, and the ability of the test compound to the modulate cancer marker's activity is determined. Determining the ability of the test compound to modulate cancer marker activity can be accomplished by monitoring, for example, changes in enzymatic activity, destruction or mRNA, viral load, or the like.

The ability of the test compound to modulate cancer marker binding to a compound, e.g., a cancer marker substrate or modulator, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

In some embodiments, the present invention provides therapies for cancer (e.g., lymphoma, leukemia or breast cancer). In some embodiments, therapies directly or indirectly target cancer markers (e.g., HERV-K(HML-2) target).

In some embodiments, the present invention targets the production of cancer markers (e.g., HERV-K(HML-2). For example, in some embodiments, the present invention employs compositions comprising oligomeric antisense or RNAi compounds, particularly oligonucleotides (e.g., those identified in the drug screening methods described above), for use in modulating the function of nucleic acid molecules encoding cancer markers of the present invention (e.g., HERV-K(HML-2), ultimately modulating the amount of cancer marker expressed.

In some embodiments, RNAi is utilized to inhibit HERV-K(HML-2) target function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g., 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, RNAi oligonucleotides are designed to target the HERV-K(HML-2) proteins.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually synthesized using a physical barrier (mask) by stepwise addition of each base in the sequence. Thus the arrays represent a full oligonucleotide complement of a region of the target gene. Hybridisation of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In some embodiments, HERV-K(HML-2) protein expression is modulated using antisense compounds that specifically hybridize with one or more nucleic acids encoding cancer markers of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent tumor proliferation.

In some embodiments, specific nucleic acids are targeted for antisense. “Targeting” an antisense compound to a particular nucleic acid, in the context of the present invention, is a multi-step process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a cancer marker of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A few genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference. Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₁ alkenyl and alkynyl. Particularly preferred are O[(CH₂)ₙO]ₘCH₃, O(CH₂)ₙOCH₃, O(CH₂)ₙNH₂, O(CH₂)ₙ—CH₃, O(CH₂)ₙONH₂, and O(CH₂)ₙON[(CH₂)ₙCH₃)]2, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

The present invention contemplates the use of any genetic manipulation for use in modulating the expression of cancer markers of the present invention. Examples of genetic manipulation include, but are not limited to, gene knockout (e.g., removing a HERV-K(HML-2) gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter (e.g., an androgen-responsive promoter)).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

In some embodiments, the present invention provides antibodies that target tumors that express a cancer marker of the present invention (e.g., HERV-K(HML-2) or associated target proteins). Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a cancer marker of the present invention (e.g., HERV-K(HML-2)), wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technetium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, .alpha.-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted a cancer marker of the present invention (e.g., HERV-K(HML-2)). Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

The present invention further provides pharmaceutical compositions (e.g., comprising pharmaceutical agents that modulate the expression or activity of HERV-K(HML-2) of the present invention). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

In some embodiments, the present invention provides compositions, kits, and methods for managing patient care. For example, in some embodiments, a diagnostic test that detects the presence of, or amount of, a HERV-K(HML-2) marker is conducted before an appropriate therapy is applied (i.e., test, then treat). In some embodiments, HERV-K(HML-2) markers are detected after treatment to monitor the success of the treatment and allow the treating physician to alter the treatment (e.g., change the compound, change the dose, discontinue, etc.) if needed or desired (i.e., treat and test, which in some embodiments, involves testing, then treating, then testing). In some embodiments, depending on the outcome of the diagnostic test, therapy is altered (i.e., treat, test, treat).

### EXPERIMENTAL

### Example 1

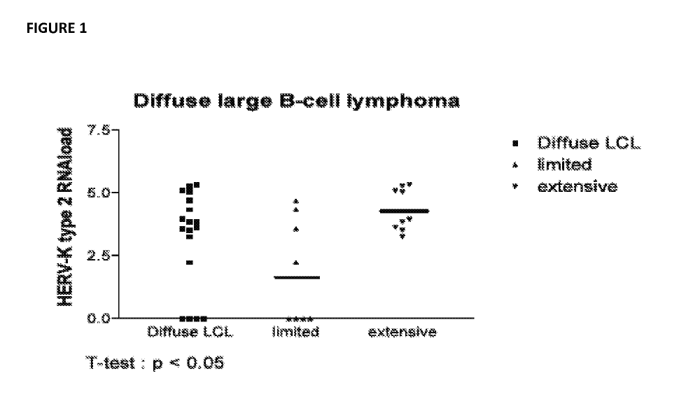

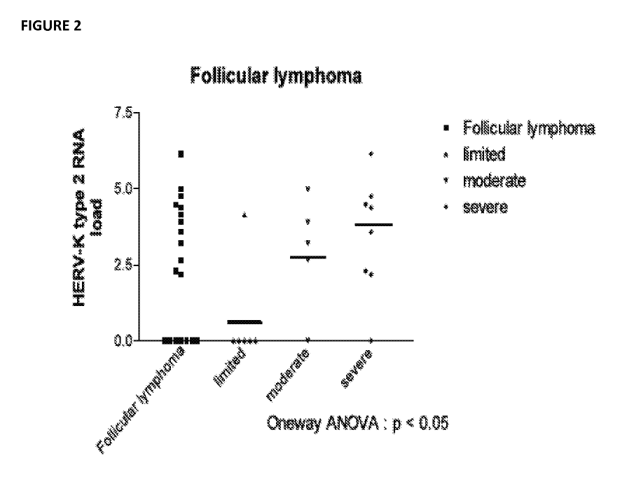

Plasma samples were collected from newly diagnosed lymphoma patients. Subjects with chronic lymphocytic leukemia were not included. Samples were obtained from over 150 patients with new onset lymphoma. HERV K (HML2) was measured in each samples using quantitative RT PCR assay that measures gag viral RNA (SEE FIG. 1). This assay indicates that in untreated lymphoma there is a considerable level of free HERV K (HML2) in plasma with non HIV associated DLCBL and HD having the highest levels of virus while patients with follicular lymphoma have somewhat lower levels of virus. The RT PCR does not distinguish type 1 from type 2 HERV K (HML2). A nucleic acid sequence based amplification assay (NASBA) was developed which allows type 1 and type 2 env to be distinguished in plasma. The assay was applied to a subset of the FL patients and to patients with DLBCL. Patients with FL with disease limited to isolated nodes and skin lesions had lower levels of viremia than those who, on bone marrow examination, were found to have lymphoma cells in the marrow as judged by flow based assays and or by immunocytogenetic analysis (SEE FIG. 2).

Levels of antibody to the Rec protein were examined. Rec is only be made by actively replicating virions. Patients with DLCBL, and HD had rather high levels of antibody to this protein while those with FL had slightly lower levels in contrast to normal patients who donated plasma samples who rarely had antibody to.

The high levels of endogenous virus in plasma is similar to the recent studies of an epidemic of lymphoma in the Australian Koala bear. East coast Koalas have been dying of lymphoma. Scientists studying these animals have discovered a new retrovirus virus called KoRv. This virus appears to have become endogenized in the Koala in the past century from a virus similar to the Gibbon ape leukemia virus (GALV). Koalas that have this endogenous virus in their genome at birth develop progressive KoRv viremia as they age and at the peak of viremia develop an aggressive often fatal lymphoma. The prolonged KoRv viremia that occurs in the Koala prior to onset of lymphoma is similar to the prolonged HERV K (HML2) viremia that we have documented in some HIV lymphoma patients in whom we have been able to measure HERV K (HML2) viral load years before the onset of lymphoma. This indicates that HERV K (HML2) becomes more infectious as time progresses causing a gradual rise in viral load and that in both HIV lymphoma and non HIV lymphoma, some recombinant or some new virus which arises through complementation may form in these plasmas which now begins to have oncogenic potential and infectious potential.

The Hamster CHO cell line can become infected with plasma associated virus from lymphoma patients.

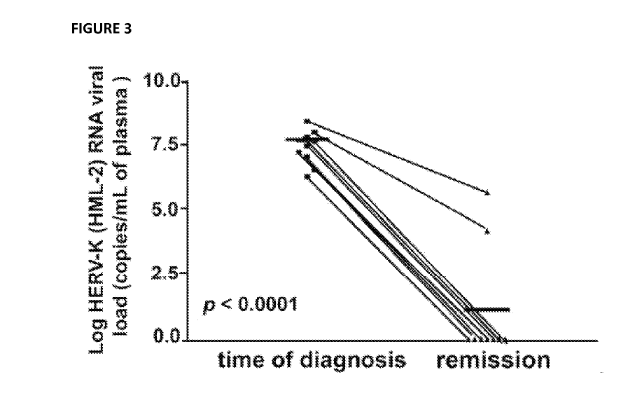

HIV associated lymphoma is dramatically reduced by highly active antiretroviral therapy (HAART). This phenomenon suggests that improved immunity as a result of HAART allows for better immune surveillance of cancer cells and or better control of EBV and HHV8. This phenomenon further indicates that one or more retroviruses ((e.g., HERV-K(HML-2) has a causative effect on lymphoma. Control of HIV TAT may also be important in reducing oncogenic risk. Experiments performed during development of embodiments of the present invention indicate that the antivirals that treat HIV, especially the nucleoside reverse transcriptase inhibitors, also have an effect on the replicative capacity of HERV K (HML2) and thereby indirectly reduce the activity of these viruses. Data indicated that these viruses play a role in lymphoma oncogenesis, and that antivirals against HERV K (HML2) reduce the risk of development of lymphoma and improve survival from lymphoma. Patients treated for lymphoma might show a reduction of the HERV K (HML2) viral load with successful lymphoma treatment. It has been demonstrated in a small group of HIV patients with different lymphomas that there was a marked drop of the HERV K (HML-2) viral load commensurate with successful cancer chemotherapy (SEE FIG. 3).

HIV antivirals were assayed to determine which antiviral agents have an antiviral effect against HERV K (HML2). The NCCIT cell line derived from a teratocarcinoma produces many HERV K (HML2) viral particles. NCCIT cells were maintained at 40% confluence in 6-well plates in RPMI medium and incubated for 7 days in the presence of increasing doses of nucleoside or non-nucleoside reverse transcriptase inhibitors or HIV protease inhibitors. Drugs, provided as lyophilized powders by the AIDS Research and Reference Reagent Program, were resuspended to a final concentration of 10 mg/mL (except for PFA: 60 mg/mL) in different solvents as recommended (SEE table 1). Cells were incubated for 7 days at increasing doses of drugs or the vehicle of solution as controls. Supernatants were collected and cell debris was removed by centrifugation at 2300 rpm for 20 min. Supernatant was assessed for Reverse Transcriptase Activity using the Reverse Transcription Assay Kit (Invitrogen) as described by the manufacturer. In addition, supernatants were treated with 20 units of DNAse (Roche) for 1 hour at 37° C. and viral RNA was extracted using the Viral RNA mini kit (Qiagen) as described by the manufacturer. The HERV-K type 2 viral load was measured by quantitative Real Time RT-PCR using primers that expand the type-2 env gene region, which is absent in type-1 viruses, Kenv type2F: 5′-AGA CAC CGC AAT CGA GCA CCG TTG A-3′ (SEQ ID NO. 1), and Kenv type2R: 5′-ATC AAG GCT GCA AGC AGC ATA CTC-3′ (SEQ ID NO. 2). Standard curves were generated using serial dilutions of in vitro RNA transcripts as external calibrators. In a similar way, quantities of HERV—K type 2 proviruses were measured by Real Time PCR using 500 ng of isolated DNA.

TABLE 1

| | |
|---|---|
| AZT (Zidovudine) | PBS |
| FTC (Emtricitabine) | PBS |
| 3TC (Lamivudine ) | PBS |
| ddC (zalcitabine) | DMSO |
| d4T (Stavudine) | PBS |
| ddI (Dideoxyinosine) | DMSO |
| TDF (Tenofovir disoproxyl fumarato) | PBS |
| ABC (Abacavir) | DMSO |
| β-d hydroxy cytidine | DMSO |

Non-Nucleoside Revene Transcriptase Inhibitors (NNRTIs)

| | |
|---|---|
| Efavirenz | DMSO |
| Nevirapine | DMSO |
| Etravirine | Acetone |

Protease Inhibitors (PIs)

| | |
|---|---|
| Atazanavir | DMSO |
| Ritonavir | DMSO |
| Indinavir | PBS |
| Amprenavir | DMSO |

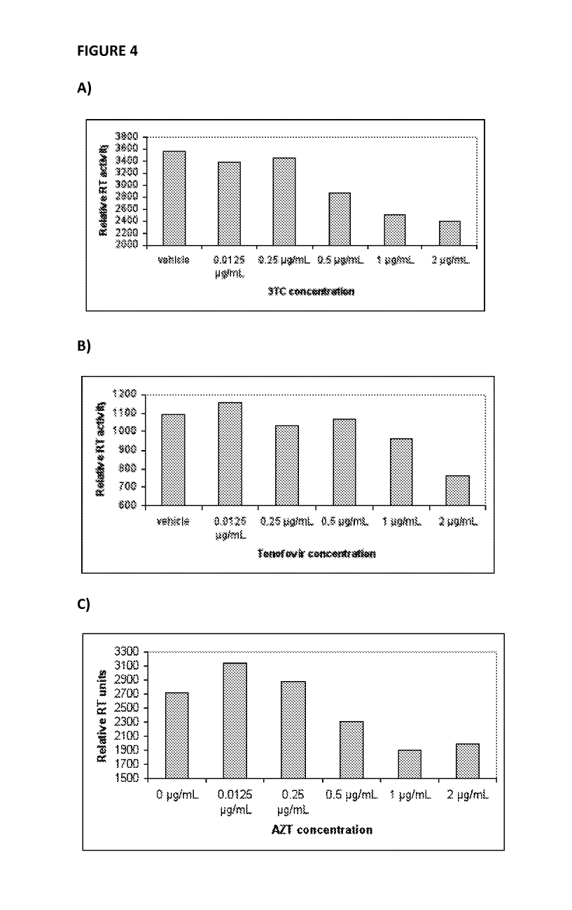

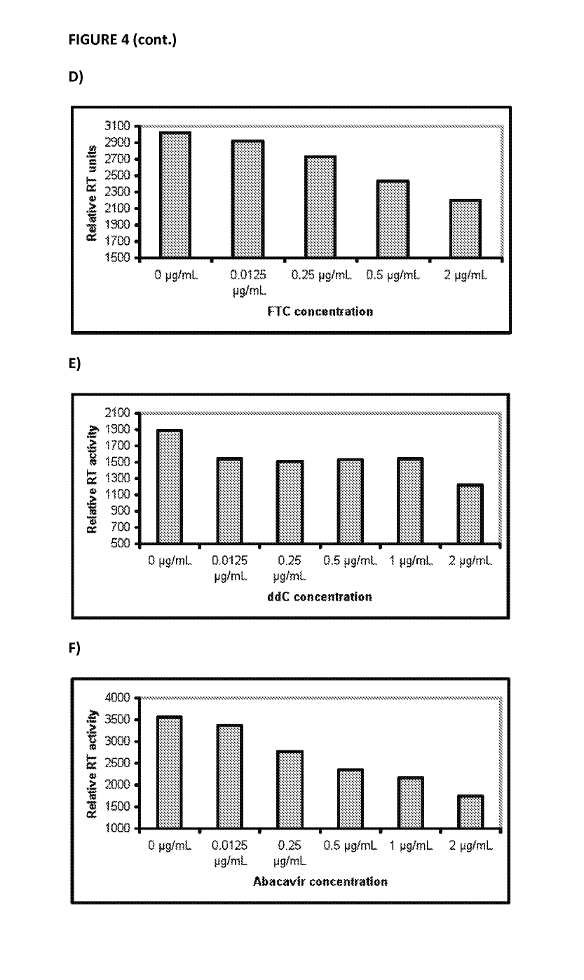

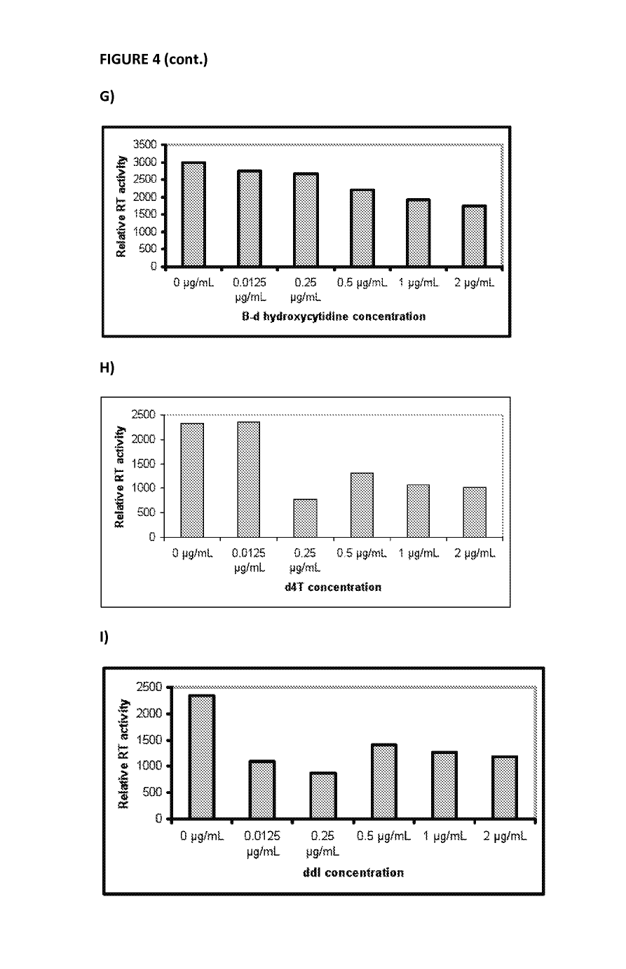

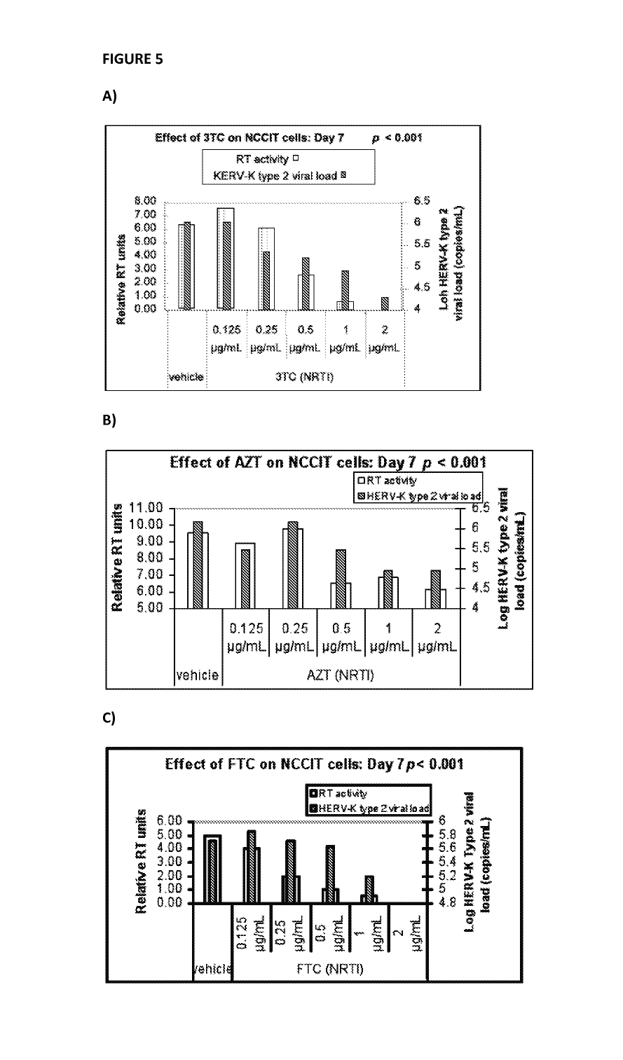

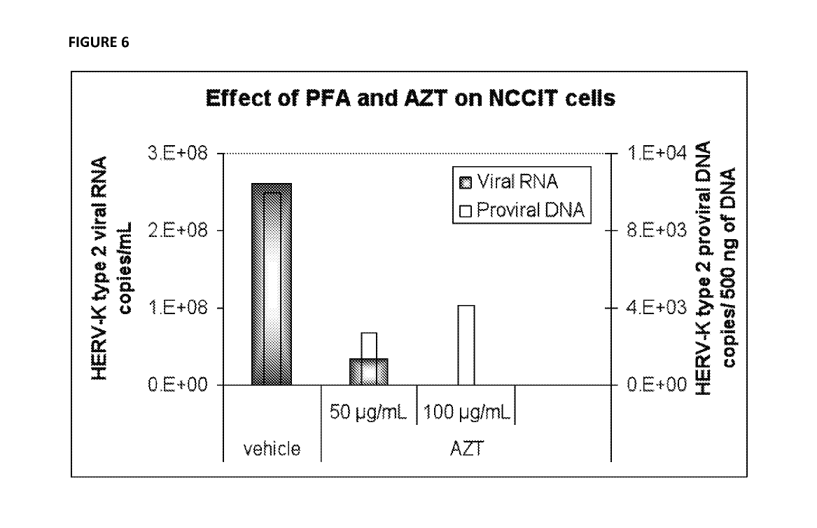

The NRTIs produced reduction in the HERV K (HML2) RT activity as shown for lamivudine and tenofovir disoproxil, AZT, FTC, ddC, Abacavir, β-D hydroxycytidine, d4T, and ddI (SEE FIGS. 4A-I). The other agents notably azidothymidine, didianosine, emtricitabine abacavir and stavudine all had activity while there was no activity from the NNRT is medications and or the protease or entry inhibitors. These latter drugs which have been designed for specific viral targets would not be expected to have activity against HERV K (HML2). However, it is contemplated that other protease inhibitors, entry inhibitors, and non-nucleoside inhibitors may demonstrate activity. In addition, HERV K-HML2 Viral RNA was also reduced (SEE FIG. 5).

NRTI antivirals can be given to patients with lymphoma to reduce the viral load of HERV K (HML2) in plasma and provide an antitumor effect on lymphoma which would demonstrate a causative role for HERV K (HML2) viruses in lymphoma.

### Example 2

Plasma samples were collected from patients who developed diffuse large B cell lymphoma as a complication of HIV infection before and after the diagnosis of lymphoma. RNA extracted from the plasma samples using the QIAamp Viral RNA Mini Kit (Qiagen, Inc. Valencia, Calif.) was subjected to RT-PCR using env-specific primers antecedent to sequencing the RT-PCR products. Genotypic trees assembled by comparing env sequences from plasma samples to known HERV K HML-2 retrovirus sequences within the human genome revealed patient specific genotypes comprising HML2 Type 1 or Type 2 viral sequences, and/or recombinant sequences between Type 1 and Type 1 viruses, Type 2 and Type 2 viruses, and/or Type 1 and Type 2 viruses. Accordingly, env sequences obtained from plasma samples find use to identify competent viruses indicative of HERV K HML2 replication and the presence of lymphoma. In some embodiments, plasma samples are subjected to detection or analysis (e.g., sequencing) using, for example, beads, microarrays, pores, and other solid and fluid high-throughput sequencing formats and platforms, or other analysis technique.

### REFERENCES

The following references, and all reference above, are herein incorporated by reference in their entireties:
Oken, et al. Am J Clin Oncol 5:649-655, 1982.
Karnofsky et al. Cancer. 1948; 1:634-656.
Painter et al. Curr Top Med Chem. 2004; 4(10):1035-44.
Lee et al. Cancer Res. 59:5514-5520, 1999
Gill et al. N Engl J Med 332:1744-1748, 1995.
Harrington et al. Lancet 1996:348; 833.
Ruprecht et al. Nature 1986:323; 467-469.
White et al. Leukemia and Lymphoma, 2001 40:287-294.
Patel et al. Ann Int Med 2008:148 728-736.
Kurokowa et al. Blood 2005:106: 235-240.
Aboulafia et al. AIDS Patient Care and STDs 2007:21:900-907
Armstrong et al. J Oral Maxillofac. Surg. 2007:65: 1361-1364
Fatkenheuer et al. Eur J Med Res 2000 5:236-240.
Al-Yamany et al. J Neurooncol 1999 42:151-9
Contreras-Galindo et al. AIDS Res Hum Retroviruses. 2006 10:979-84.
Kaplan et al. Amer J Med 82(3):389-396, 1987.
Contreras-Galindo et al. J. Virology 82(19):9329-9336, 2008.
Thio et al. AIDS Reviews 2007:9:40-53.
Levy et al. Clinical Infectious Diseases 2006:43; 904-910
Marcellin et al. N Engl J Med 2008 359:2442-2455.
Tarlington et al. Mol. Lif Sci 2008 65:3413-3421.
Thelader et al. Leukemia and Lymphoma 2008; 49:1042-49.
Tempescul A et al. Ann Hematol. Published on line.
Brody et al. Critcal Rev Oncology Hematol: 2006; 58257-265.
Zelenetz et al. Ann Onc 2006:17 (supplement 4) Lv12-lv14.
Moller et al. Brit J Hematol 2006; 133:43-49.
Tan et al. Hematol Oncol Clin N Am 2008 22:863-882.
Tilly et al. Leukemia and Lymphoma 2008; 49(S1):7-17.
DePaepe et al. Leukemia 2006 1-7.
Friedberg et al. Hematol Oncol Clin N Am 2008; 22:941-52.
Smith et al. Curr Opin Hematol 2008; 15:415-421.
Shakir et al. J Clin Pahtol 2008; 61:920-927.
Zhou et al. Cancer 2008; 113:791-8.
Peinert et al. Hematol Oncol Clin N Am 2008 22:903-940.
Leleu et al. European J of Hematol 2008; 82:1-12.
Sargent et al. Am J Surg Pathol 2008: 32:1643-1653.

We claim:

1. A method for treating cancer comprising treating a subject suffering from cancer with one or more compounds sufficient to reduce the viral load of HERV K (HML-2).

2. The method of claim 1, wherein said cancer comprises lymphoma.

3. The method of claim 2, wherein said lymphoma comprises HIV-associated lymphoma.

4. The method of claim 2, wherein said lymphoma comprises non-HIV-associated lymphoma.

5. The method of claim 1, wherein said subject does not suffer from HIV infection.

6. The method of claim 1, wherein said compounds comprise antiretroviral pharmaceuticals.

7. The method of claim 6, wherein said antiretroviral pharmaceuticals comprise reverse transcriptase inhibitors.

8. The method of claim 7, wherein said reverse transcriptase inhibitors are selected from nucleoside analog reverse transcriptase inhibitors and nucleotide analog reverse transcriptase inhibitors.

9. The method of claim 1, wherein reducing said viral load of HERV K (HML-2) causes a reduction in tumor burden.

10. The method of claim 1, wherein reducing said viral load of HERV K (HML-2) eliminated said HERV K (HML-2) viruses from said subject.

11. The method of claim 1, wherein HERV K (HML-2) is detected in a sample from said subject prior to, during, or following treatment.

12. The method of claim 11, wherein treatment choice is selected based on said detection.

13. A method of screening compounds useful in the treatment of cancer comprising screening compounds for usefulness in reducing viral load of HERV K (HML-2).

14. The method of claim 13, wherein said screening is performed in vitro.

15. The method of claim 13, wherein said screening is performed in vivo.

16. The method of claim 13, wherein said screening comprises administering one or more said compounds to cells and assaying cells for a reduction in viral load of HERV K (HML-2).

17. The method of claim 13, wherein said screen comprises high throughput screening.

18. The method of claim 15, wherein said compounds are further assayed for usefulness in treating cancer.

19. The method of claim 13, wherein said cancer comprises lymphoma.

20. The method of claim 19, wherein said lymphoma comprises HIV-associated lymphoma.

21. The method of claim 19, wherein said lymphoma comprises non-HIV-associated lymphoma.

22. The method of claim 13, wherein said compounds comprise antiretroviral pharmaceuticals.

23. The method of claim 22, wherein said antiretroviral pharmaceuticals comprise reverse transcriptase inhibitors.

24. The method of claim 23, wherein said reverse transcriptase inhibitors are selected from nucleoside analog reverse transcriptase inhibitors, nucleotide analog reverse transcriptase inhibitors, and non-nucleoside reverse transcriptase inhibitors.


SEQUENCE LISTING (2 sequences)

SEQ ID NO: 1 - DNA, 25 bp, Artificial Sequence
  Synthetic
agacaccgcaatcgagcaccgttga

SEQ ID NO: 2 - DNA, 24 bp, Artificial Sequence
  Synthetic
atcaaggctgcaagcagcatactc